(19) United States
(12) Patent Application Publication
(10) Pub. No.: US 2025/0279183 A1
(43) Pub. Date: Sep. 4, 2025

(54) Strategy For Positive Impact

(71) Applicant: Paresh B Fozdar, Elmhurst, NY (US)

(72) Inventor: Paresh B Fozdar, Elmhurst, NY (US)

(21) Appl. No.: 18/517,678

(22) Filed: Nov. 22, 2023

(51) Int. Cl. G16H 20/70

(52) U.S. Cl. CPC G16H 20/70

(57) ABSTRACT

Strategy For Positive Impact is applicable in any field for positive impact, such as essential conduct for balance with progress and growth in habitat. Application of Strategy For Positive Impact on need point out wrong, such as question by example on behavior and anomaly indicate harm in environment: exploitation such as toxic bias and immorality, misfire on survival; outplay system, such as perpetration and misdirection, for deterioration. Iteration of Strategy For Positive Impact on wrong identify right action for capable, such as analysis and inquiry on harm with observation derive true conduct for balance: guard for moral rights such as spot misconduct, hoax and bias in power for punishment; aid for viable creative such as consensus on passion with ability for integrity and trust, with effort in honest direction; growth for advancement, such as inheritance of diversity and availability of factual, towards believable and virtuous progress.

### APPROACH

Formulate strategy to produce positive impact by nomenclature: Strategy For Positive Impact (SPI)

Apply SPI, to identify need with interpretation and derive positive impact on society and environment, as proof

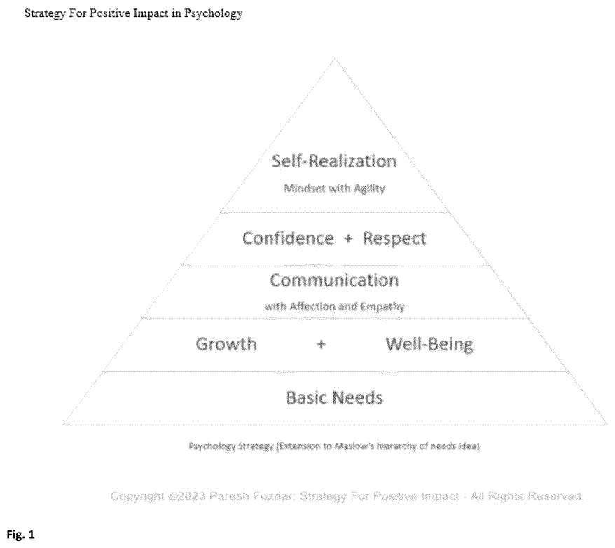

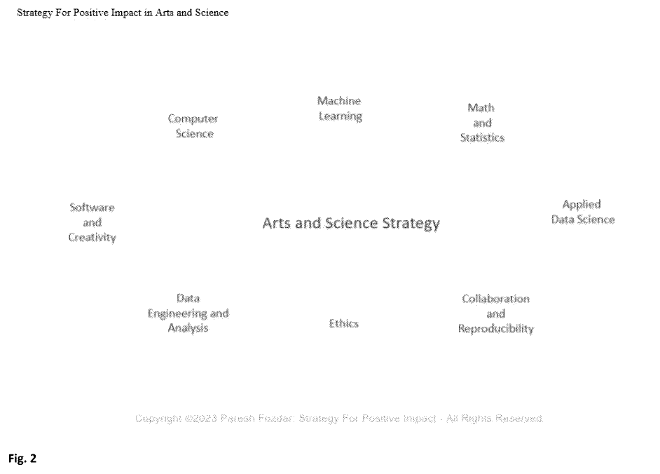

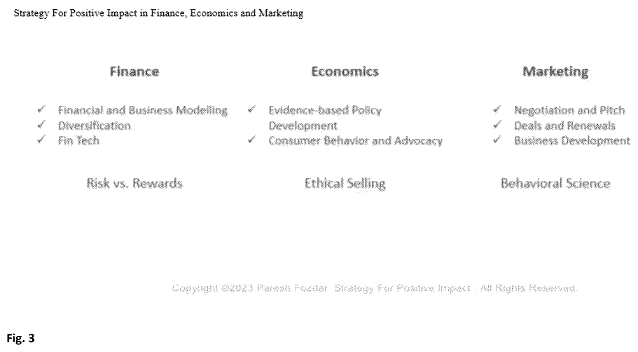

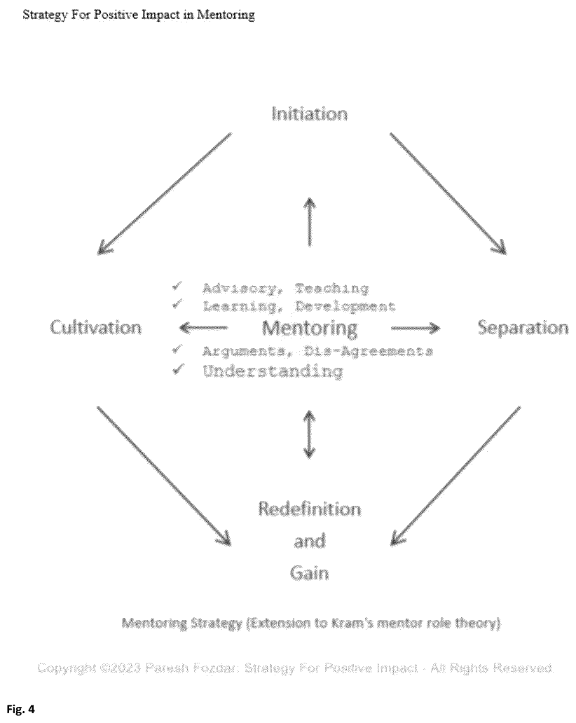

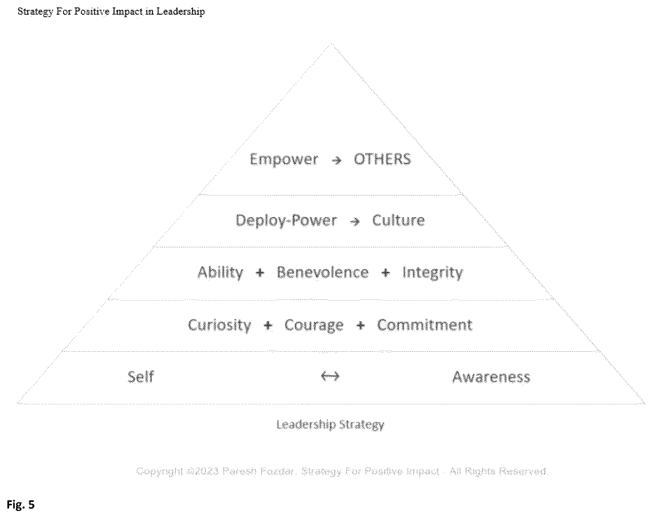

For SPI in
Psychology:
   Refer to, FIG. 1 is SPI in Psychology
Arts and Science:
   Refer to, FIG. 2 is SPI in Arts and Science
Finance, Economics and Marketing:
   Refer to, FIG. 3 is SPI in Finance, Economics and Marketing
Mentoring:
   Refer to, FIG. 4 is SPI in Mentoring
Leadership:
   Refer to, FIG. 5 is SPI in Leadership

### COMPONENTS

Need
Explanation
Impact

### NEED

Apply SPI to analyze behavior and anomaly by question

### BEHAVIOR

Fear
excessive attempt to converse
fight loneliness
solitude for avoidance
seclusion with withdrawal
Thrive
sacrifice of interest
achieve goal by external coercion
take advantage from position of social privilege
impair ability with divide
Augment
interpret to grasp lifeform
comprehend to classify egocentricity
predict on basis of thought and activity
exploitation by bias
Strive
mask evil in self
conceal toxic for contamination
utilize insult as shield, to accomplish
Embody
stun to propagate misery
posture reactively to resolve, for gain
develop untrue belief by characteristics, for stereotype trap

### ANOMALY

Acclaim
recognize immorality to defer
research arrangement for evident or obvious
amplify fallacy with loyalty anchor
Sprout
obscure requirement with unreasonable expectation
persevere by belief to profile
retain advantage by growth sham
adopt archaic grief or method to detain
obstruct by no synergy to hinder
Burgeon
bias accrue by echelon to embrace power
support for perpetrator by legitimate power
misdirect invest or expertise
Exhaust
stagnate capability by suppression of detail or data
dismiss or poach, for benefit
retain impropriety in allocation of asset
Fail
pop with strenuous defense rational
   deteriorate productivity
declare disorder or ailment for inability to solve
   evade accountability
define exclusion for write-off inclusion
   repeat or replicate for claim

### ANALYSIS

Human behavior and anomaly in system, indicate harm

### EXPLANATION

Apply SPI to inquire and learn from need by example

### EXAMPLE

Arrangement by congruity for
Psychology

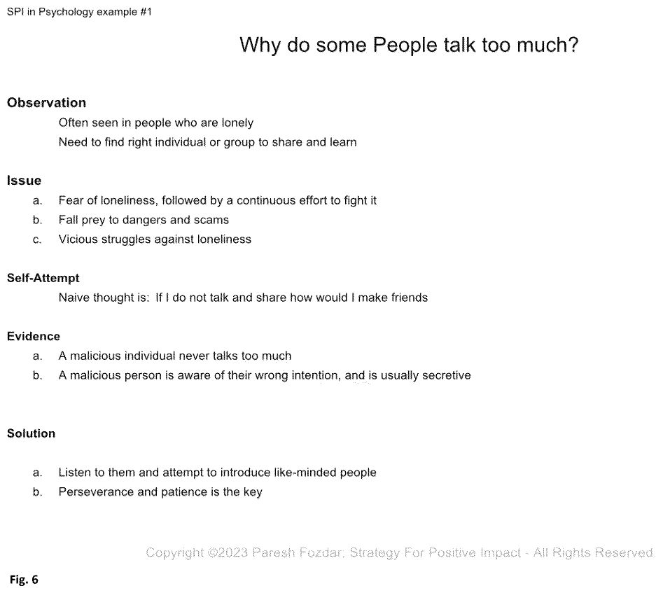

Refer to, FIG. 6 is SPI in Psychology example #1

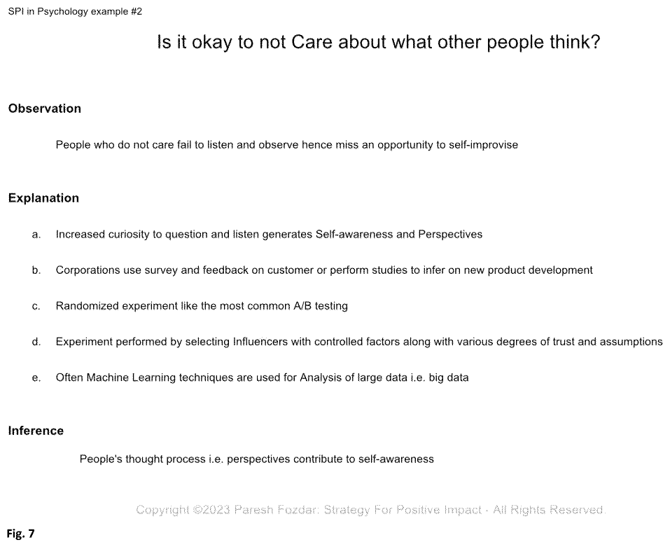

Refer to, FIG. 7 is SPI in Psychology example #2

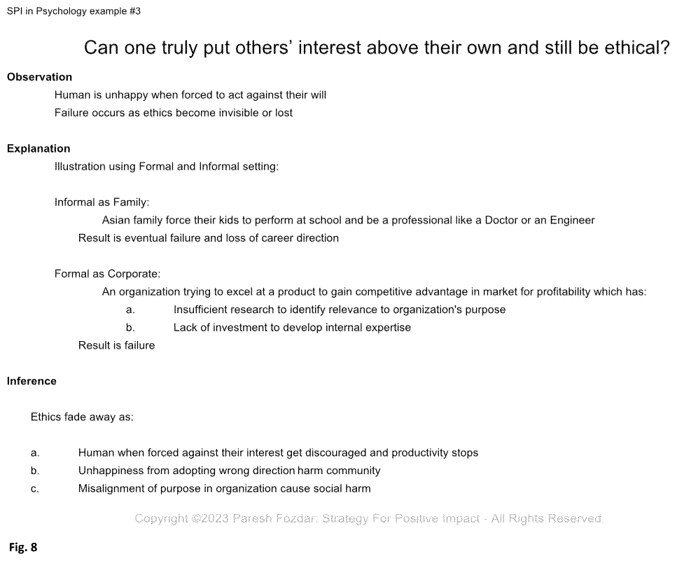

Refer to, FIG. 8 is SPI in Psychology example #3
Arts and Science

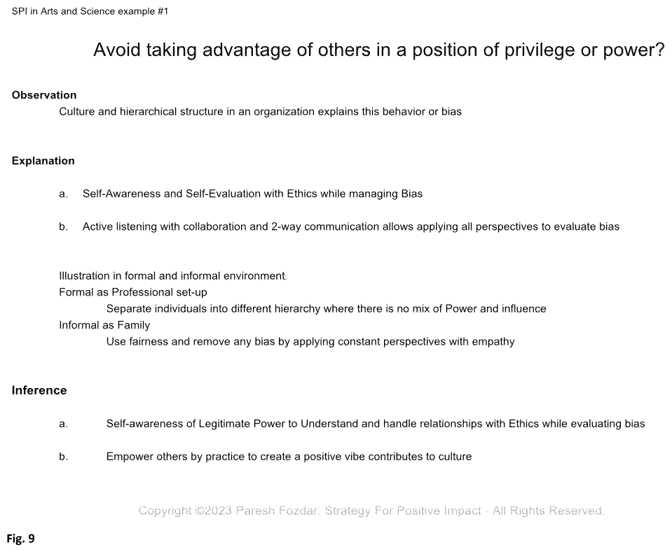

Refer to, FIG. 9 is SPI in Arts and Science example #1

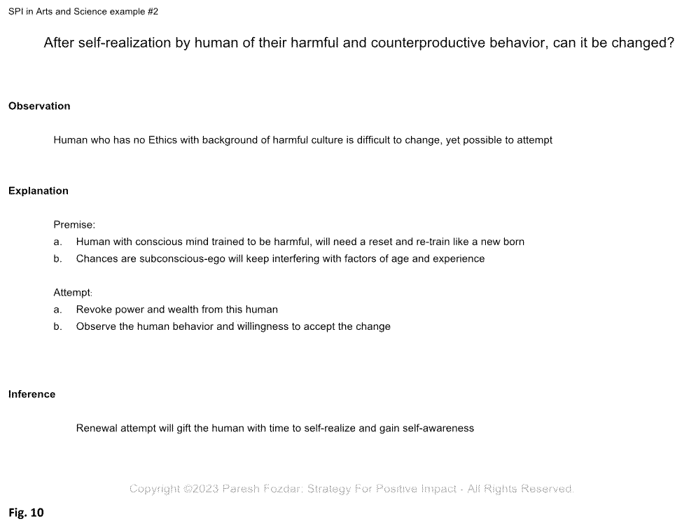

Refer to, FIG. 10 is SPI in Arts and Science example #2

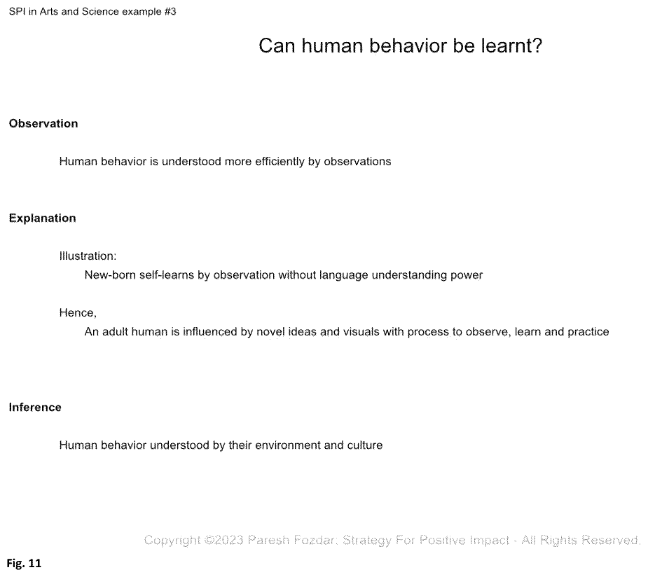

Refer to, FIG. 11 is SPI in Arts and Science example #3
Finance, Economics and Marketing

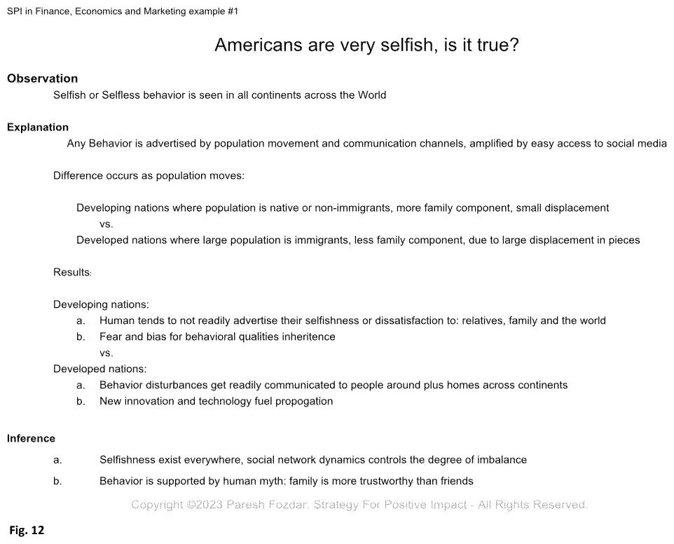

Refer to, FIG. 12 is SPI in Finance, Economics and Marketing example #1

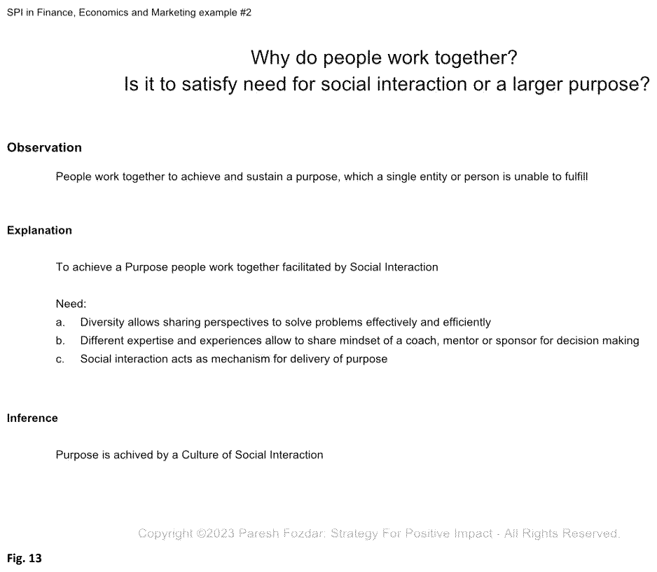

Refer to, FIG. 13 is SPI in Finance, Economics and Marketing example #2

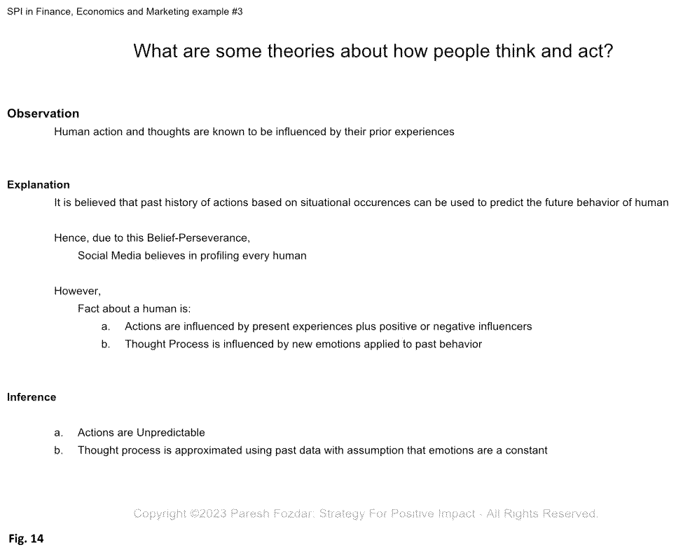

Refer to, FIG. 14 is SPI in Finance, Economics and Marketing example #3
Mentoring

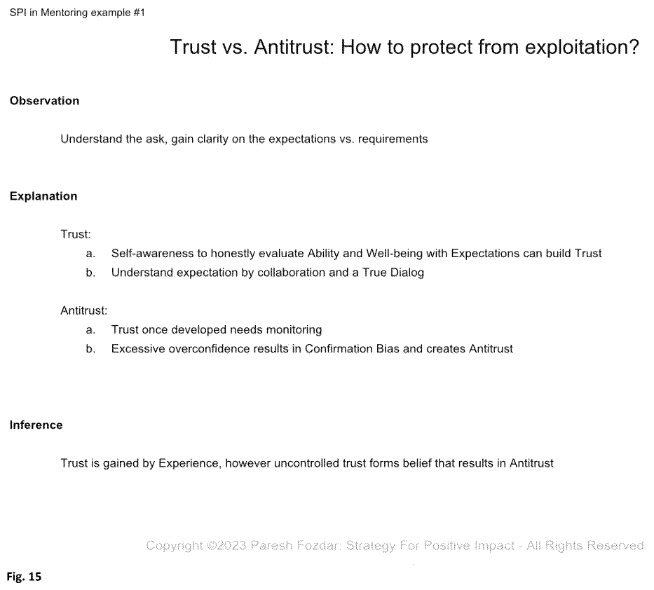

Refer to, FIG. 15 is SPI in Mentoring example #1

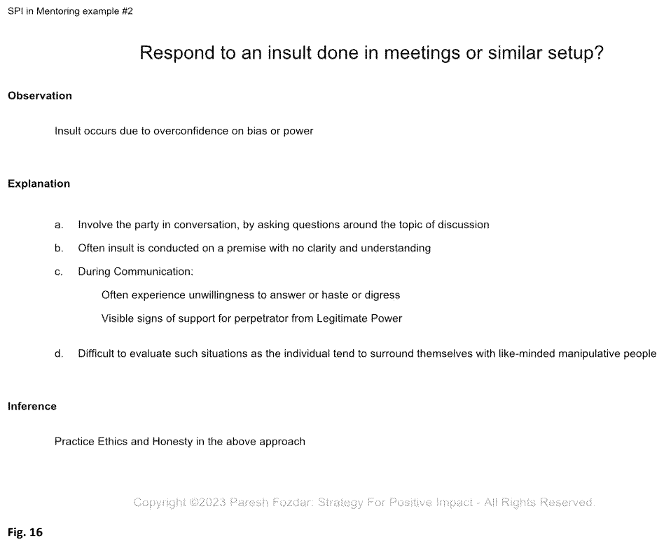

Refer to, FIG. 16 is SPI in Mentoring example #2
Leadership

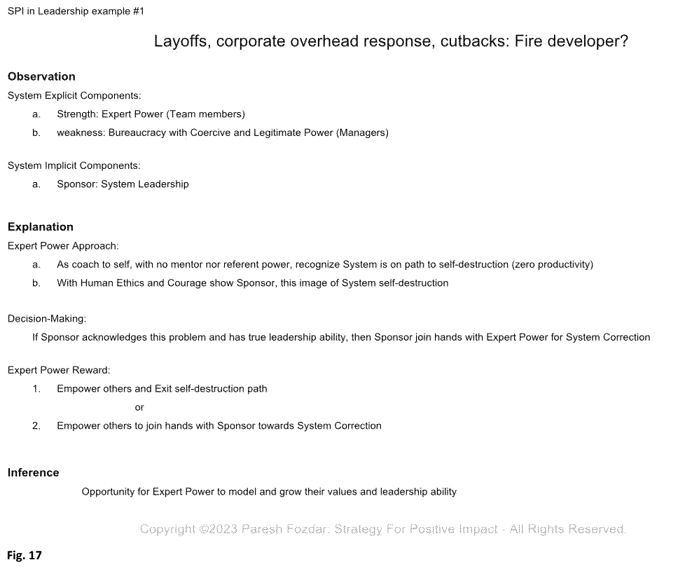

Refer to, FIG. 17 is SPI in Leadership example #1

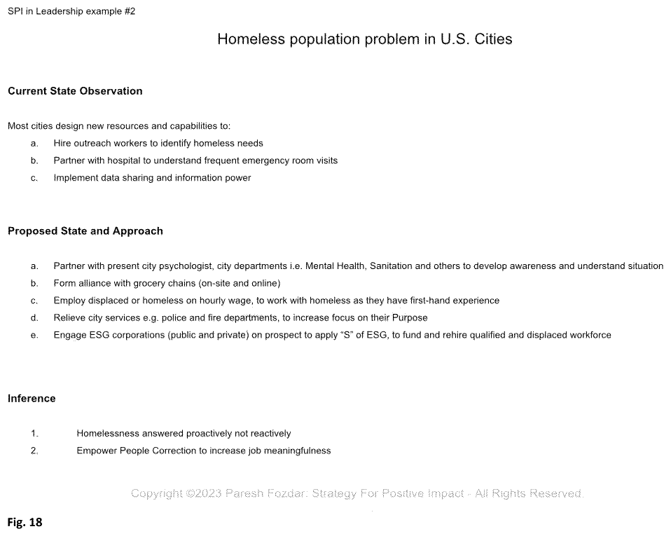

Refer to, FIG. 18 is SPI in Leadership example #2

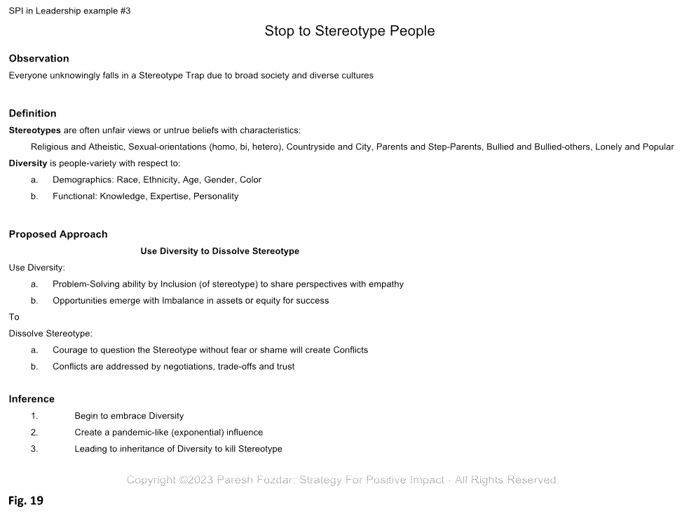

Refer to, FIG. 19 is SPI in Leadership example #3

### HARM

Observation on need point out wrong
Exploit
distribute power for imbalance
affect work with vicious domain
instigate disturbance with:
   bias by inherent menace for defamation
   fear by coercion from authority
Surrender
forgo opportunity to learn and improvise, by pressure
pamper slow decision-making by inaccessibility
lose productivity
   disconnect by misalignment of interest or expertise with goal
   manifest friction by inaptitude, and mislead extrinsic community
   rocket non-ethics by disinclination for unsuitable
Scam
hoax for adherence to myth
produce directive to develop troublesome predictive analysis
seize by appraisal of:
   communication tact
   confidence level
Fraud
pestilence of immorality
abide to bully
amass alike to crowd and coerce for acquisition
Scandal
circle to dispose talent by convolution for contortion, to exhibit bureaucracy
exploit with overconfidence and belief, for bias as norm
replicate nonfunctional by stratum with collusion, for distress

### POSITIVE

Analyze observation to identify and strengthen righteous
Upbeat
endeavor to fight and achieve goal
develop perspective for awareness
existence of social obedience
Respect
pitch to predict movement or speculate, is futile
ignorance and negligence devise belief for malpractice, ensue confirmation on bias for antitrust
expertise to perceive destruction with:
   attempt to correct
   empower and exit
Effort
organism indoctrinate
   learn by percept
   understand by environment
   spot attribute for misconduct: haste, digression, idiocy, oblivion
simplify with reproducibility to deliver by: diversity, expertise, agile-mindset
propose alternate with proactive approach to raise efficiency and effectiveness
Vitalize
advance art for benevolent-inventful.
   use as tool: machine to learn, software to assist
fuel science for balance
   employ math and statistics for simplification
   engineer for productive
   analyze data for examples to derive information and context
Ripple
develop evaluation for bias by: listen, collaboration
accept viewpoint with affinity, to nurture trust
   curiosity to overcome fear
   courage to inquire
   negotiate to resolve conflict

### ACTION

Apply SPI for inquiry on positive
Train
facilitate experience or disagreement, for trust
listen, spot and prevent attack from malicious
discipline wrong by revocation of power
   realize by renewal attempt
ease availability of factual data for fulfilment
embrace to inherit diversity and kill stereotype
Empower
foster awareness:
   explore perspective
   interpret or discuss thought process for experimental
cultivate creative:
   reward and redeem for novel
   extend meaningfulness
gear emotion component as prerequisite for change
enable expert to model leader capability
Deploy
propel ethics and honesty for progress
equity for well-being
   distribute influence and power
   use fairness by perspective with empathy
   monitor dependence for moderation to believe
support virtuous for integrity
   align passion, interest and purpose

### CONDUCT

Continual advance with well-being, by ethical advocacy, mentoring and leadership, for creative and prosperous growth

Application of SPI, to assess harm and reinforce positive with commitment by action for impact

### IMPACT

Iterative application of SPI with analysis on need and inquiry on harm, identifies positive conduct to realize essential balance in habitat

### STATEMENT FOR NO NEW MATTER

This Statement confirms that this Substitute Specification contains no new matter, except addition, brief description of drawings

1. Strategy For Positive Impact is applicable in any field for positive impact, such as essential conduct for balance with progress and growth in habitat.

2. Application of Strategy For Positive Impact on need point out wrong, such as question by example on behavior and anomaly indicate harm in environment:
a. Exploitation such as toxic bias and immorality, misfire on survival;
b. Outplay system, such as perpetration and misdirection, for deterioration.

3. Iteration of Strategy For Positive Impact on wrong identify right action for capable, such as analysis and inquiry on harm with observation derive true conduct for balance:
a. Guard for moral rights such as spot misconduct, hoax and bias in power for punishment;
b. Aid for viable creative such as consensus on passion with ability for integrity and trust, with effort in honest direction;
c. Growth for advancement, such as inheritance of diversity and availability of factual, towards believable and virtuous progress.
